(19) United States
(12) Patent Application Publication
(10) Pub. No.: US 2010/0173912 A1
(43) Pub. Date: Jul. 8, 2010

(54) Novel Compound

(76) Inventors: Alessandro Artioli, Essex (GB); Peter John Lovell, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/663,752

(22) PCT Filed: Jun. 12, 2008
(86) PCT No.: PCT/EP2008/057374

§ 371 Date: Dec. 9, 2009

(30) Foreign Application Priority Data
Jun. 14, 2007 (GB) 0711521.5

(51) Int. Cl. A61K 31/495; C07D 295/192; A61P 25/18

(52) U.S. Cl. 514/255.01; 544/391

(57) ABSTRACT

The invention provides 5-fluoro-2-[4-({[(4-fluorophenyl)methyl]oxy}acetyl)-1-piperazinyl]benzonitrile of formula (I),

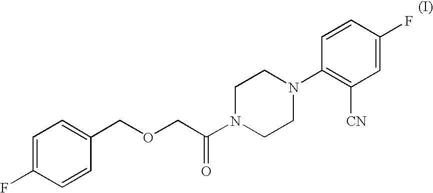

or a pharmaceutically acceptable salt thereof

The invention relates to piperazine derivatives for treating diseases and conditions mediated by positive allosteric modulation of the G-protein coupled metabotropic subtype 5 receptor (mGluR5), such as neurological and psychiatric disorders, for example schizophrenia. In addition, the invention relates to compositions containing the derivatives and processes for their preparation.

Glutamate is the major excitatory neurotransmitter in the nervous system and exerts its action through both ligand-gated ion channels and G-protein coupled metabotropic receptors (mGluR). Evidence suggests that mGlu5 receptors are generally located at post-synaptic levels and they are mainy distributed in limbic forebrain areas. Aberrant glutamatergic neurotransmission (due to excessive activation of glutamate receptors and abnormalities in glutamate receptor binding and mRNA expression), has been implicated in several neurological and psychiatric disorders. In particular, modulation of glutamatergic neurotransmission to alleviate a hypoglutamatergic state existing in schizophrenia has received increasing support over the past few years and may provide a complementary approach to traditional dopamine based therapies.

A number of mGluR5 agonists and antagonists have been identified to date which bind at the orthosteric binding site but suffer poor sub-type selectivity due to high sequence homology in this region. It has been suggested that positive allosteric modulation of mGluR5 may provide therapeutic benefits over orthosteric modulators for the treatment of neurological and psychiatric disorders such as schizophrenia (Kinney et al, J. Pharmacol. And Exp. Ther., (2005), 313 (1), 199-206; Lindsley et al, J. Med. Chem. (2004), 47 (24), 5825-8). Due to the conserved ligand binding domain within the mGluR family, allosteric modulators should provide greater selectivity over orthosteric modulators as the 7 transmembrane domain is less conserved between mGluR subtypes. In addition agonists binding to the orthosteric binding site have been suggested to be pro-convulsant (Chapman et al, Neuropharmacol. (2000), 39 (9), 1567-74), algesic (Walket et al, Neuropharmacol (2001), 40 (1), 1-9), neurotoxic (Blaabjerg et al (2001) 898 (1), 91-104) and anxiogenic (Perez de la Mora et al Eur. J. Neurosci. (2006) 23 (10), 2749-59), making allosteric modulation of the receptor a more attractive proposition. Allosteric modulators require the presence of an endogenous agonist and are saturable so should mimic the normal physiological effects of the endogenous ligand which suggests the effects of a patient taking an overdose of an allosteric modulator may be less than an overdose of an orthosteric modulator

It is therefore an object of the invention to provide a compound for treating diseases and conditions mediated by positive allosteric modulation of the G-protein coupled metabotropic subtype 5 receptor (mGluR5), such as neurological and psychiatric disorders, for example schizophrenia.

Therefore, according to a first aspect, the invention provides 5-fluoro-2-[4-({[(4-fluorophenyl)methyl]oxy}acetyl)-1-piperazinyl]benzonitrile of formula (I)

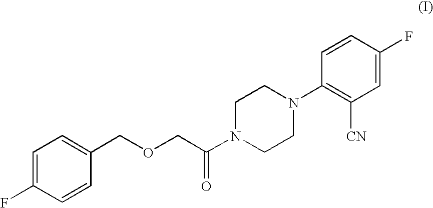

or a pharmaceutically acceptable salt thereof.

The compound of formula (I) may form pharmaceutically or veterinarily acceptable salts, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Hereinafter, the compound of formula (I) and its pharmaceutically acceptable salts, are referred to as “ the compounds of the invention”.

It will be appreciated by those skilled in the art that certain protected derivatives of the compounds of the invention, which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compound defined in the first aspect.

The compounds of the invention may exist in solvated or hydrated form.

The compounds of the invention or solvates/hydrates of the compounds or salts, may exist in one or more polymorphic forms.

Therefore, according to a further aspect, the invention provides a solvate, hydrate or prodrug of the compounds of the invention.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

As discussed above, it is considered that positive allosteric modulation of mGluR5 may treat neurological and psychiatric disorders, for example schizophrenia.

Therefore, according to an embodiment, the invention provides a compound of the invention for use as a medicament, preferably a human medicament.

According to a further aspect, the invention provides the use of a compound of the invention in the manufacture of a medicament for treating or preventing a disease or condition mediated by positive allosteric modulation of mGluR5.

In an embodiment, diseases or conditions that may be mediated by positive allosteric modulation of mGluR5 are selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).

ii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).

iii) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

iv) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

v) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

vi) Enhancement of cognition including the treatment of cognition impairment in other diseases such as schizophrenia, bipolar disorder, depression, other psychiatric disorders and psychotic conditions associated with cognitive impairment, e.g. Alzheimer's disease.

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is suitably presented as a pharmaceutical formulation.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

In addition the compounds of the invention may be administered in combination with 5-HT₃ antagonists (such as ondansetron, granisetron and metoclopramide); serotonin agonists (such as sumatriptan, rauwolscine, yohimbine and metoclopramide); and NK-1 antagonists.

It will be appreciated that the compound of the combination or composition may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions.

The compounds of the invention will normally, but not necessarily, be formulated into pharmaceutical compositions prior to administration to a patient by an appropriate route. Accordingly, in another aspect, the invention provides pharmaceutical compositions comprising a compound of the invention and one or more pharmaceutically-acceptable excipients.

As used herein, “pharmaceutically-acceptable excipient” means any pharmaceutically acceptable material present in the pharmaceutical composition or dosage form other than the compound or compounds of the invention. Typically the material gives form, consistency and performance to the pharmaceutical composition.

The pharmaceutical compositions of the invention typically contain one compound of the invention. However, in certain embodiments, the pharmaceutical compositions of the invention contain more than one compound of the invention. In addition, the pharmaceutical compositions of the invention may comprise one or more additional pharmaceutically active compounds.

Such pharmaceutical compositions of the invention may be prepared and packaged in bulk form wherein a safe and effective amount of a compound of the invention can be dispensed and then given to the patient such as with powders or syrups. Alternatively, the pharmaceutical compositions of the invention may be prepared and packaged as dosage forms wherein each physically discrete dosage form contains a safe and effective amount of a compound of the invention. Accordingly, in another aspect, the invention provides dosage forms comprising pharmaceutical compositions of the invention. Each discrete dosage form contains from 1 mg to 500 mg of a compound of the invention. In another aspect, each discrete dosage form contains from 5 mg to 400 mg of a compound of the invention. In another aspect, each discrete dosage form contains from 10 mg to 300 mg of a compound of the invention. In another aspect, each discrete dosage form contains from 20 mg to 300 mg of a compound of the invention.

It will be recognised by one of skill in the art that the optimal quantity and spacing of individual dosages of compounds of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of compounds of the invention given per day for a defined number of days, can be ascertained by those skilled in the art using conventional course of treatment determination tests.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the compound of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the compounds of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

It will be appreciated that the invention includes the following further aspects. The diseases and conditions described above extend, where appropriate, to these further aspects.
i) A compound of the invention for use in treating or preventing a disease or condition mediated by positive allosteric modulation of mGluR5.
ii) A method of treatment or prevention of a disease or condition mediated by positive allosteric modulation of mGluR5 in a mammal comprising administering an effective amount of a compound of the invention.

### Preparation of 5-fluoro-2-[4-({[(4-fluorophenyl)methyl]oxy}acetyl)-1-piperazinyl]benzonitrile

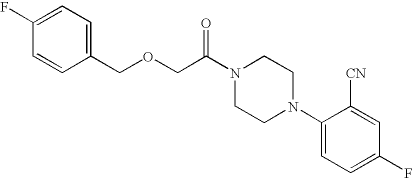

A mixture of {[(4-fluorophenyl)methyl]oxy}acetic acid (Intermediate 1) (2.96 g, 16.07 mmol) and 5-fluoro-2-(1-piperazinyl)benzonitrile (Intermediate 2) (3.63 g, 17.68 mmol) in N,N-dimethylformamide (35 ml) and diisopropylethylamine (5.61 ml, 32.1 mmol) was stirred at room temperature. HATU (7.03 g, 18.48 mmol) was added portionwise. The mixture was stirred at room temperature for 10 minutes and the then allowed to stand at room temperature overnight. The reaction mixture was concentrated under reduced pressure and the residue partitioned between ethyl acetate and half saturated sodium bicarbonate solution. The organic layer was separated, washed with water, brine, dried (MgSO₄) and concentrated under reduced pressure. The crude compound was purified by column chromatography on silica gel (40+M) eluting with ethyl acetate in isohexane (5% ethyl acetate to 80% ethyl acetate in isohexane). The clean fractions were combined and concentrated under reduced pressure to give pale yellow oil which was triturated with petrol/ether to give the title compound a white solid (2.99 g).

¹H NMR (400MHz, DMSO-d6) δ (ppm): 7.76 (1H, dd, J=8.4 Hz, 3.2 Hz), 7.50-7.56 (1H, m), 7.40-7.43 (2H, m), 7.16-7.24 (3H, m), 4.52, (2H, s), 4.26 (2H, s), 3.60 (4H, m), 3.07 (4H, br s). m/z 372 [M+H]⁺

### Intermediate 1: {[(4-fluorophenyl)methyl]oxy}acetic acid

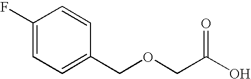

A solution of 4-fluorobenzyl alcohol (9.34 g, 74.1 mmol) in THF (20 ml) was slowly added to a suspension of sodium hydride (3.56 g, 89 mmol) in THF (45 ml) over a period of 45 minutes at room temperature. The mixture was then stirred at room temperature for 10 minutes. Chloroacetic acid (3.5 g, 37.0 mmol) in THF (20 ml) was slowly added over 15 minutes, precipitation occurred. The mixture was heated at 60° C. overnight (17 hours) under a flow of argon. The mixture was allowed to cool to room temperature and water (100 ml) was added. The mixture was washed with ethyl acetate (×3), the aqueous phase acidified with 5M HCl and extracted with ethyl acetate (×3). The combined organics were dried (MgSO₄) and concentrated under reduced pressure to give 4.9 g of pale yellow oil. NMR analysis showed the presence of chloroacetic acid. The oil was dissolved in diethyl ether and washed with water (×4). The organic layer was dried (MgSO₄) and concentrated under reduced pressure to give the title compound as a very pale yellow oil (2.96 g).

¹H NMR (400 MHz, DMSO-d6), δ (ppm) 12.7 (1H, br s), 7.39 (2H, m), 7.18 (2H, m), 4.51 (2H, s), 4.06 (2H, s). m/z: 183 [M−H].

### Intermediate 2: 5-fluoro-2-(1-piperazinyl)benzonitrile

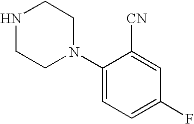

A mixture of piperazine (0.50 g, 5.8 mmol) and 2,5 difluorobenzonitrile (0.83 g, 6 mmol) in NMP (8 mL) was heated in a microwave reactor at 150° C. for 15 minutes. LC/MS analysis showed partial conversion to the target, so the mixture was heated again using the microwave reactor at 150° C. for an additional 10 minutes. The mixture was passed through an SCX column and the product collected by elution with 2M ammonia in methanol. The solvents were removed by evaporation to afford a yellow oil, this was redissolved in dichloromethane and filtered. Removal of the solvent by evaporation afforded the title compound as a yellow oil (0.72 g).

¹H NMR (400 MHz, CDCl₃), δ (ppm) 7.28 (1H, dd), 7.23 (1H, m), 7.01 (1H, m), 3.10 (8H, m). m/z [M+H]⁺206

In the procedures above, after each starting material, reference to an intermediate is often provided. This is provided merely for assistance to the skilled chemist. The starting material may not necessarily have been prepared from the batch referred to.

Compounds are named using ACD/Name PRO 6.02 chemical naming software (Advanced Chemistry Development Inc., Toronato, Ontario, M5H2L3, Canada).

Proton Magnetic Resonance (NMR) spectra were recorded either on Varian instruments at 300, 400 or 500 MHz, or on a Bruker instrument at 300 or 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is reported.

Mass spectra (MS) were taken on a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS−ES (+): analysis performed on an Waters Atlantis column (50×4.6 mm); stationary phase particle size 3 uM; mobile phase A: aqueous phase=water+0.05% formic acid; mobile phase B=organic solvent=acetonitrile+0.05% formic acid. Method as follows:

| Time/min | % A | % B |
|---|---|---|
| 0 | 97 | 3 |
| 0.1 | 97 | 3 |
| 4 | 3 | 97 |
| 4.8 | 3 | 97 |
| 4.9 | 97 | 3 |
| 5.0 | 97 | 3 |

The above method has a flow rate of 3 mL/min. The injection volume is 5 uL. The column temperature is 30 deg C. The UV detection range is from 220 to 330 nm.

For reactions involving microwave irradiation, a Biotage Initiator was used.

Flash silica gel chromatography was carried out on silica gel 230-400 mesh (supplied by Merck AG Darmstadt, Germany) or over pre-packed Biotage silica cartridges.

SPE-SCX cartridges were supplied by Varian. The eluent used with SPE-SCX cartridges was methanol followed by 2N ammonia solution in methanol.

In a number of preparations purification was performed using either Biotage manual flash chromatography (Flash+) or automatic flash chromatography (Horizon) systems. All these instruments work with Biotage Silica cartridge.

SPE-Si cartridges were supplied by Varian.

Abbreviations

The following table lists the used abbreviations:
DMSO Dimethylsulphoxide
HATU—(O-7-azabenzotriazol-1-yl)-N,N,N′,N′-tetramethyluronium hexafluorophosphate)
NMP—N-Methylpyrrolidinone
THF—Tetrahydrofuran

Biological Assay

Primary Cortical Astrocyte Culture

Primary glial cultures were prepared from cortices of Sprague Dawley (CD1—cesarian derived) rats—postnatal day 2. The cortices were dissected and then dissociated by trituration in Dulbecco's Modified Eagle's Medium (D-MEM containing 1000 mg/L glucose, Invitrogen) supplemented with papain (30 U/mL, Worthington Laboratories), 0.24 mg/mL L-cysteine (Sigma), 40 μg/mL DNAse I type IV (Sigma). Papain treatment was stopped with D-MEM supplemented with 1 mg/mL trypsin inhibitor (Sigma), 50 μg/mL BSA (Sigma) and 40 μg/mL DNAse I type IV (Sigma). The resulting cell homogenate was plated onto T175 flasks—pre-coated with poly-D-lysine (MW: >300,000, Sigma) in D-MEM supplemented with 15% heat-inactivated fetal bovine serum (FBS), 2.2 mL glucose, penicillin and streptomycin and incubated at 37° C. and 5% CO₂. 3 days after plating the media was replaced with fresh growth medium (as above). 3 days later the medium was replaced with D-MEM supplemented with 10% heat-inactivated fetal bovine serum (FBS), 2.2 mL glucose, penicillin and streptomycin for 6 hours. The flasks were then shaken vigorously overnight at 37° C. to remove oligodendrocytes leaving astrocytes adhered to the flasks. The next day cells were subplated by trypinisation onto poly-D-lysine precoated 384-well plates at a density of 10,000 cells per well in D-MEM supplemented with 10% dialysed heat-inactivated fetal bovine serum (FBS), 2.2 ml glucose, penicillin and streptomycin and growth factors (basic fibroblast growth factor (5 ng/mL) and epidermal growth factor (10 ng/mL) incubated at 37° C. and 5% CO2 for 48 hours prior to assaying. Under exposure to growth factors rat cultured astrocytes express increased levels of mGlu5 receptors.

CHO cells Expressing Human mGluR5b

CHO cells expressing human mGluR5b (pSwitch vector, Invitrogen) were maintained in DMEM media with 10% Foetal Calf Serum, 0.005% Hygromycin, Proline(10 mg/mL), Zeocin(0.005%). The media was replaced between subculturing days to remove any excess glutamate build up that may cause receptor desensitization. 24 hrs prior to conducting the assay, cells were plated in 384-well black flat clear bottom plates and mGluR5 expression was induced with the addition of 0.01 nM mifepristone. The plates were incubated at 37° C. to give a monolayer with a confluency of 80%.

FLIPR Assay

The tissue culture medium was aspirated using a Tecan power washer. Cells were loaded with 30 μl of Hanks Balanced Salts (HBSS)+2.5 M Probenicid+2 μM Fluo-4+250 μM Brilliant Black+0.01% Pluronic acid. The cells were incubated at 37° C. for 2 hours to allow uptake of the dye Fluo-4AM into the cell cytosol, which was converted to Fluo-4 by natural esterases cleaving the AM region preventing the Fluo-4 leaving the cell.

Compound Preparation

The compound to be tested was prepared by dissolving in DMSO at a concentration of 10 mM. These solutions were further diluted with DMSO using a Biomek FX (Beckman Coulter) in a 384 well compound plate. Each dilution was transferred in 1 μL aliquots to a further compound plate and assay buffer (HBSS+2.5 M Probenicid) was added to a volume of 50 μL, making a final assay concentration of 11.8 μM in the assay.

Estimation of ˜EC20 Concentration of Glutamate(Endogenous Ligand)

A 0.38 M solution of glutamic acid was prepared in water. This was further diluted in DMSO to a concentration of 255 mM. A range of concentrations of glutamate were prepared in assay buffer and dispensed using a Multidrop(Thermolabsystems) into a 384 well compound plate. The chosen standard positive modulator was prepared in a 384 well compound plate at a concentration of 1 mM in DMSO. A Biomek FX was used to produce a serial dilution in DMSO. 1 μL of each dilution was transferred to a further compound plate where it was diluted in 50 μL of assay buffer. This gives a final top assay concentration of 4 μM. 10 μL of this compound plate was added ‘on line’ on FLIPR to a loaded cell plate. A fluorescent reading was made. The cells with compound added were incubated at 37° C. for 10 min after which a plate containing the range of glutamate concentrations was added to the cells in a second ‘on line’ FLIPR addition. A second fluorescent read was made. A visual choice of ˜EC20 concentration was made from the raw data generated. The chosen concentration of glutamate was dispensed into 384 well compound plates using a Multidrop and was high enough to allow modulation to be viewed without being so high that it masked modulation.

Running the Assay

The compound was added ‘on line’ to the cells and a first FLIPR read was made to estimate agonism. The cell plates were then incubated at 37° C. for 5-10 min after which the chosen EC20 Glutamate was added ‘on line’ and a second FLIPR reading made to estimate modulation. Modulation potency was normalised to EC20 Glutamate response as basal and EC100 Glutamate response as total.

5-fluoro-2-[4-({[(4-fluorophenyl)methyl]oxy}acetyl)-1-piperazinyl]benzonitrile showed activity in the human mGluR5b positive modulator assay with a pEC50 value of greater than 6.0.

What is claimed is:

1. 5-Fluoro-2-[4-({[(4-fluorophenyl)methyl]oxy}acetyl)-1-piperazinyl]benzonitrile of formula (I) 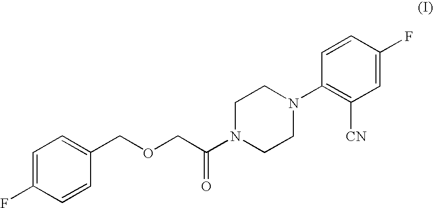
or a pharmaceutically acceptable salt thereof.

2-6. (canceled)

7. A method of treating a psychotic disorder in a human in need thereof comprising administering to said human a therapeutically effective amount of a compound as defined in claim 1 or a pharmaceutically acceptable salt thereof.

8. The method as claimed in claim 7, wherein the psychotic disorder is schizophrenia.

9. A pharmaceutical composition for the treatment of psychotic disorders comprising a compound as defined in claim 1 or a pharmaceutically acceptable salt thereof.
